(19) United States
(12) Patent Application Publication
(10) Pub. No.: US 2006/0128616 A1
(43) Pub. Date: Jun. 15, 2006

(54) Process for the purification of tnf-binding proteins using imac

(75) Inventor: Mara Rossi, Rome (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: Ares Trading S.A., Aubonne (CH)

(21) Appl. No.: 10/534,535

(22) PCT Filed: Nov. 13, 2003
(86) PCT No.: PCT/EP03/50824

§ 371 Date: Sep. 8, 2005

(30) Foreign Application Priority Data
Nov. 15, 2002 (EP) 02025755.6

(51) Int. Cl. A61K 38/17; C07K 14/715; C07K 16/24

(52) U.S. Cl. 514/12; 530/350

(57) ABSTRACT

A new purification process for Tumor Necrosis Factor-binding proteins is described. In particular this process is characterized by the use as capture step of an Immobilized Metal Affinity Chromatography (IMAC) using copper as metal. This brings advantages in terms of process yields, purity of the final product and applicability to industrial scale.

### FIELD OF THE INVENTION

This invention relates to the field of polypeptide purification. More specifically, it relates to the purification of Tumor Necrosis Factor-binding proteins.

### BACKGROUND OF THE INVENTION

Tumor necrosis factor-alpha (TNFA), a potent cytokine, elicits a broad spectrum of biologic responses which are mediated by binding to a cell surface receptor. The receptor for human TNF-alpha may be isolated from a human histiocytic lymphoma cell line (see Stauber et al., J. Biol. Chem., 263, 19098-104, 1988).

Using monoclonal antibodies, another group obtained evidence for 2 distinct TNF-binding proteins, both of which bind TNF-alpha and TNF-beta specifically and with high affinity (see Brockhaus et al., Proc. Nat. Acad. Sci. 87: 7380-7384, 1990) and isolated the cDNA for one of the receptors. They found that it encodes a protein of 455 amino acids that is divided into an extracellular domain of 171 residues and a cytoplasmic domain of 221 residues.

Later on another group (see Aggarwal et al. Nature 318: 665-667, 1985) showed that tumor necrosis factors alpha and beta initiate their effects on cell function by binding to common cell surface receptors. The TNF alpha and TNF beta receptors have different sizes and are expressed differentially in different cell lines (see Engelmann et al., J. Biol. Chem. 265: 1531-1536, 1990).

TNF alpha Receptor I, referred to by some as TNFR55, is the smaller of the 2 receptors. cDNAs for both receptors have been cloned and their nucleic acid sequence determined (see Loetscher et al., Cell 61: 351-359, 1990; Nophar et al., EMBO J. 9: 3269-78, 1990; Schall et al., Cell 61: 361-370, 1990 and Smith et al., Science 248: 1019-1023, 1990).

Whereas the extracellular domains of the 2 receptors are strikingly similar in structure, their intracellular domains appear to be unrelated. Southern blotting of human genomic DNA, using the cDNAs of the 2 receptors as probes, indicated that each is encoded by a single gene.

Several approaches have been attempted to purity polypeptides. Chromatography is one of the means most commonly used, including affinity chromatography in which the substance to be purified is first adsorbed to a bed or column of a suitable support on which agents having affinity for the given substance are immobilized to capture it and let the remaining components of the raw mixture pass unbound. The adsorbed substance is then eluted by changing such environmental conditions as pH and/or salt concentration to give a partially or totally purified molecule.

In the field of affinity chromatography, the technique known as IMAC (Immobilized Metal Affinity Chromatography) has been described as particularly efficient in certain cases (see the review article by Arnold, Biotechnology, Vol. 9, page 151-156, February 1991). IMAC is described as a powerful technique in the purification of polypeptides having functional groups that participate in metal binding, such as the side chains of Glu, Tyr, Cys, His, Asp and Met, as well as the amino-terminal amide nitrogens and backbone carbonyl oxygens.

Although the technique is powerful, it does not always have the required specificity. For example, it has been ascertained that adsorption on a Cu²⁺ containing chromatographic column is excellent for polypeptides containing one or preferably more histidines, but it was also observed that even in the absence of the three amino-acids considered to be most important for adsorption, namely histidine, tryptophan and cysteine, adsorption of protein may occur, thus impairing the specificity of the purification step.

The adsorption efficiency, although generally satisfactory for purification purposes, may not be optimal particularly when the polypeptide to be purified is a glycoprotein. In this case very often the carbohydrate chains may conceal the sites active for the binding to the metal chelate and reduce the affinity for the chromatographic column in the adsorption step.

### DESCRIPTION OF THE INVENTION

It has now been found that TNF-binding proteins can be efficiently purified by means of a process including an Immobilized Metal Affinity Chromatography (IMAC) step using copper as metal. Optimal conditions of pH and salinity for this step are a pH of 2.8 to 3.2, preferably pH 3, and a salinity of 14 to 16 mS, preferably of 15 mS.

According to the present invention “TNF-binding proteins” means any protein which has an affinity for TNF-alpha or TNF-beta and/or a protein which comprises in the extra-cellular, soluble fragment of a protein belonging to the TNF receptors family, or a fragment thereof.

Some examples of members of the TNF receptor family are the following:
Tumor Necrosis Factor Receptor 1 (TNFR1), also called Tumor Necrosis Factor Receptor Superfamily, Member 1A (TNFRSF1A), or Tumor Necrosis Factor-alpha Receptor (TNFAR) or TNFR 554(D or TNFR 60-KD (see description at OMIM*191190 http://www.ncbi.nlm.nih.gov/entrez/query.fcgi?db=OMIM)>
Tumor Necrosis Factor Receptor 2 (TNFR2), also called Tumor Necrosis Factor Receptor Subfamily, Member 1B (TNFRSF1B), or Tumor Necrosis Factor-beta Receptor (TNFBR) or TNFR 75-KD or TNFR 804(D (see description at OMIM*191191);
OX40 Antigen (OX40), also called Tumor Necrosis Factor Receptor Superfamily, Member 4 (TNFRSF4), or Tax-Transcriptionally Activated Glycoprotein 1 Receptor (TXGP1L) or Lymphoid Activation Antigen ACT35 (ACT35) or CD134 (see description at OMIM*600315);
CD40L Receptor (CD40), also called Tumor Necrosis Factor Receptor Superfamily, Member 5 (TNFRSF5) or B-cell surface antigen CD40, or CDw40 or Bp50 (see description at Swiss-Prot Entry No. P25942);
FASL Receptor (FAS), also called Tumor Necrosis Factor Receptor Superfamily, Member 6 (TNFRSF6), or Apoptosis-Mediating Surface Antigen FAS or Apo-1 Antigen or CD95 (see description at Swiss-Prot Entry No. P25445);
Decoy Receptor 3 (DcR3), also called Tumor Necrosis Factor Receptor Superfamily, Member 6B (TNFRSF6B) or Decoy Receptor for FAS Ligand or M68 (see description at Swiss-Prot Entry No. O95407);
CD27 Atnigen (CD27), also called Tumor Necrosis Factor Receptor Superfamily, Member 7 (TNFRSF7) or T-Cell Activation Antigen S152 (S152) (see description at OMIM*602250);
Lymphoid Activation Antigen CD30 (CD 30), also called Tumor Necrosis Factor Receptor Superfamily, Member 8 (TNFRSF8) (see description at OMIM*153243)
Induced By Lymphocyte Activation (ILA), also called Tumor Necrosis Factor Receptor Superfamily, Member 9 (TNFRSF9) or CD137 (see description at OMIM-602250);
Death Receptor 4 (DR4), also called Tumor Necrosis Factor Receptor Superfamily, Member 10A (TNFRSF10A), or TNF-Related Apoptosis-inducing Ligand Receptor 1 (TRAILR1) or APO2 (see description at OMIM*603611);
Death Receptor 5 (DR5), also called Tumor Necrosis Factor Receptor Superfamily, Member 10B (TNFRSF10B), or TNF-Related Apoptosis-Inducing Ligand Receptor 2 (TRAILR2) or killer/DR5 or TRICK2 (see description at OMIM*603612);
Decoy Receptor 1 (DCR1), also called Tumor Necrosis Factor Receptor Superfamily, Member 10C (TNFRSF10C), or TNF-Related Apoptosis-inducing Ligand Receptor 3 (TRAILR3), or TRAIL Receptor Without An Intracellular Domain (MID) (see description at OMIM*803613);
Decoy Receptor 2 (DCR2), also called Tumor Necrosis Factor Receptor Superfamily, Member 10D (TNFRSF10D) or TNF-Related Apoptosis-including Ligand Receptor 4 (TRAILR4) or TRAIL Receptor With A Truncated Death Domain (TRUNDD) (see description at OMIM*603014);
Receptor Activator of NF-KAPPA-B (RANK), also called Tumor Necrosis Factor Receptor Superfamily, Member 11A (TNFRSF11A), or Osteoclast Differentiation Factor Receptor (ODFR) or PDB2 or TRANCER (see description at OMIM*803499);
Osteoprotegerin (OPG), also called Tumor Necrosis Factor Receptor Superfamily, Member 11B (TNFRSF11B) or Osteoclastogenesis Inhibitory Factor (OCIF) (see description at OMIM*602643);
Death Receptor 3 (DR3), also called Tumor Necrosis Factor Receptor Superfamily, Member 12 (TNFRSF12), or APO3 or Lymphocyte-Associated Receptor of Death (LARD) (see description at OMIM*603366);
Transmembrane Activator And Cami Interactor (TACI), also called Tumor Necrosis Factor Receptor Superfamily, Member 13B (TNFRSF13B) (see description at OMIM*604907);
BAFF Receptor (BAFFR), also called Tumor Necrosis Factor Receptor Superfamily, Member 13C (TNFRSF13C), or B Cell-Activating Factor Receptor (see description at OMIM*606269);
Herpesvirus Entry Mediator (HVEM), also called Tumor Necrosis Factor Receptor Superfamily, Member 14 (TNFRSF14), or Herpesvirus Entry Mediator A (HVEA) or TR2 (see description at OMIM*602746);
Nerve Growth Factor Receptor (NGFR), also called Tumor Necrosis Factor Receptor Superfamily, Member 16 (TNFRSF16) or p75(NTR) (see description at OMIM*162010);
B-Cell Maturation Factor (BCMA), also called Tumor Necrosis Factor Receptor Superfamily, Member 17 (TNFRSF17) or BCM (see description at OMIM*109545);
Glucocorticoid-Induced TNFR-Related Gene (GITR), also called Tumor Necrosis Factor Receptor Superfamily, Member 18 (TNFRSF18), or Activation-Inducible TNFR Family Member (AITR) (see description at OMIM*603905);
TRADE, also called Tumor Necrosis Factor Receptor Superfamily, Member 19 (TNFRSF19), or Toxicity and JNK Inducer or TROY or TAJ (see description at Swiss-Prot Entry No. Q9NS68);
X-linked Ectodyplasin-A2 Receptor (XEDAR), also called EDA-A2 receptor (see description at Swiss-Prot Entry No. Q9HAV5) and
DEATH RECEPTOR 6 (DR6), also called Tumor Necrosis Factor Receptor Superfamily, Member 21 (TNFRSF21) (see description at OMIM*605732).

According to a preferred embodiment of the invention the TNF-binding protein is selected from recombinant h-TBP-1 (recombinant, extracellular, soluble fragment of human TNF Receptor-1, comprising the amino add sequence corresponding to the 20-180 amino acids fragment of Nophar et al.,) and recombinant h-TBP-2 (recombinant, extracellular, soluble fragment of TNF Receptor-2, comprising the amino acid sequence corresponding to 23-257 of Smith et al.). Most preferably, it is recombinant hTBP-1 (r-hTBP-1). For all the other proteins the soluble, extracellular domain is indicated in the corresponding Swiss-Prot entry.

According to another preferred embodiment of the invention, the purification process of the TNF-binding protein includes the “IMAC” step as the “capture step” and further comprise the following steps, as “Intermediate steps”: Ion exchange chromatography (IEC) at an acidic pH (preferably between 3 and 4) followed by ion exchange chromatography at a basic pH (preferably between 8 and 10).

According to a further preferred embodiment of the invention the purification process of the TNF-binding protein further comprises, as “polishing step” hydrophobic interaction chromatography (HIC).

More preferably each of the above mentioned chromatography step is followed by an ultrafiltration.

“Capture step” according to the present invention means the step during which the recombinant TNF-binding protein is isolated and concentrated from the crude harvest supernatant of the recombinant host cells culture containing it. A high yield at the end of this initial step has a big impact on the overa II performance and yield of the process. According to the present invention, the capture step carried out on Cu-Chelate FF and, preferably, with an elution at pH 3.0 yields a product having a purity >40% and a recovery >80%.

“Intermediate steps” are the steps during which most of the bulk impurities, such as other proteins and nucleic acids, endotoxins and viruses are removed.

“Polishing steps” are the steps during which any remining trace impurities or closely related substances are removed, in order to obtain a high purity protein.

“Ion exchange chromatography” (EC) is capable of separating molecules that have only slight differences in charge to give a very high resolution separation. Fractions are collected in purified, concentrated form. The separation is based on the reversible interaction between a charged molecule and an oppositely charged chromatographic medium. Molecules bind as they are loaded onto the column. Conditions are then altered so that the bound substances are eluted differentially. Elution is usually performed by changes in salt concentration or pH. Changes are made stepwise or with a continuous gradient Q Sepharose or SP Sepharose columns are commonly used in ion exchange chromatography. “Q Sepharose” is a quaternary ammonium strong anion exchanger (charged groups: —N⁺(CH₃)₃), whereas “SP Sepharose” is a sulfopropyl strong cation exchanger (charged groups: —SO₃⁻)

Hydrophobic interaction chromatography (HIC) is a versatile method for the purification and separation of biomolecules based on differences in their surface hydrophobicity. Proteins and peptides usually sequester hydrophobic amino acids in domains away from the surface of the molecule. However, many biomolecules considered hydrophilic have sufficient hydrophobic groups exposed to allow interaction with hydrophobic ligands attached to the chromatographic matrix. Compared to reversed phase chromatography, the density of the ligand on the matrix is much lower. This feature promotes the high selectivity of HIC, while allowing mild elution conditions to help preserve biological activity. “Butyl Sepharose” column is preferably used according to the present invention in the hydrophobic interaction chromatography (HIC) step. On this column the n-butyl group is used as hydrophobic ligand.

According to the present invention, the TNF-binding proteins are produced by means of recombinant DNA technology in eukaryotic, preferably mammalian, cells. The recombinant process for producing them is here below reported for completeness.

In the initial step of the process the DNA sequence coding for the desired protein is inserted and ligated into a suitable plasmid. Once formed, the expression vector is introduced into a suitable host cell, which then expresses the vector(s) to yield the desired protein.

Expression of any of the recombinant proteins of the invention as mentioned herein can be effected in eukaryotic cells (e.g. yeasts, insect or mammalian cells) or prokaryotic cells, using the appropriate expression vectors. Any method known in the art can be employed.

For example the DNA molecules coding for the proteins obtained by any of the above methods are inserted into appropriately constructed expression vectors by techniques well known in the art (see Sambrook et al, 1989). Double stranded cDNA is linked to plasmid vectors by homopolymeric tailing or by restriction lining involving the use of synthetic DNA linkers or blunt-ended ligation techniques: DNA ligases are used to ligate the DNA molecules and undesirable joining is avoided by treatment with alkaline phosphatase.

In order to be capable of expressing the desired protein, an expression vector should comprise also specific nucleotide sequences containing transcriptional and translational regulatory information linked to the DNA coding the desired protein in such a way as to permit gene expression and production of the protein. First in order for the gene to be transcribed, it must be preceded by a promoter recognizable by RNA polymerase, to which the polymerase binds and thus initiates the transcription process. There are a variety of such promoters in use, which work with different efficiencies (strong and weak promoters).

For eukaryotic hosts, different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated.

The DNA molecule comprising the nucleotide sequence coding for the hybrid protein of the invention is inserted into vector(s), having the operably linked transcriptional and translational regulatory signals, which is capable of integrating the desired gene sequences into the host cell. The cells which have been stably transformed by the introduced DNA can be selected by also introducing one or more markers which allow for selection of host cells which contain the expression vector. The marker may also provide for phototrophy to a auxotropic host, biocide resistance, e.g. antibiotics, or heavy metals such as copper, or the like. The selectable marker gene can either be directly linked to the DNA gene sequences to be expressed, or introduced into the same cell by co-transfection. Additional elements may also be needed for optimal synthesis of proteins of the invention.

Factors of importance in selecting a particular plasmid or viral vector include: the ease with which recipient cells, that contain the vector may be recognized and selected form those recipient cells which do not contain the vector; the number of copies of the vector which are desired in a particular host; and whether it is desirable to be able to “shuttle” the vector between host cells of different species.

Once the vector(s) or DNA sequence containing the construct(s) has been prepared for expression the DNA construct(s) mat be introduced into an appropriate host cell by any of a variety of suitable means: transformation, transfection, conjugation, protoplast fusion, electroporation, calcium phosphate-precipitation, direct microinjection, etc.

Host cells may be either prokaryotic or eukaryotic. Preferred are eukaryotic hosts, e.g. mammalian cells, such as human, monkey, mouse, and Chinese hamster ovary (CHO) cells, because they provide post-translational modifications to protein molecules, including correct folding or glycosylation at correct sites. Also yeast cells can carry out post-translational peptide modifications including glycosylation. A number of recombinant DNA strategies exist which utilize strong promoter sequences and high copy number of plasmids which can be utilized for production of the desired proteins in yeast. Yeast recognizes leader sequences on cloned mammalian gene products and secretes peptides bearing leader sequences (i.e., pre-peptides).

After the introduction of the vector(s), the host cells are grown in a selective medium, which selects for the growth of vector-containing cells. Expression of the cloned gene sequence(s) results in the production of the desired proteins.

Purification of the recombinant proteins so obtained is carried out according to the method of the invention.

A very detailed embodiment of the present invention will be presented in the following part of this specification and is schematically summarized in FIG. 1.

Abbreviations

TNF Tumor Necrosis Factor
TBP TNF Binding Protein
IDA Iminodiacetic acid
Cu-Chelate FF Copper-Chelate Fast Flow
Q-SEPH. FF Q-Sepharose Fast Flow
SP-SEPH. FF SP-Sepharose Fast Row
Butyl-SEPH FF Butyl-Sepharose Fast Flow
IEC Ion Exchange Chromatography
ACN Acetonitrile
CBB Comassie Brilliant Blue
DNA Deoxyribonucleic Acid
EtOH Ethanol
HIC Hydrophobic Interaction Chromatography
IEF Iso Electric Focusing
IEMA Immuno-EnzymoMetric Assay
IFMA Immuno Fluorimetric Assay
IPC In Process Control
KD Kilo Dalon
LOQ Limit of Quantitation
OD Optical Density
PI Isoelectric Point
RP-HPLC Reverse Phase High Performance Liquid Chromatography
SDS-PAGE or SDS Sodium Dodecyl Sulphate Poly Acrylammide Gel Electrophoresis
SE-HPLC Size Exclusion High Performance Liquid Chromatography
SMW Molecular weight standards
SS Sodium Sulphate
Tris Tris(hydroxymethyl)aminomethane
BV Bed Volume

### DESCRIPTION OF THE FIGURE

FIG. 1: this figure shows a flow chart of the process used for the purification of r-hTBP-1. From the capture step up to the achievement of the r-hTBP-1 bulk material 8 steps are performed, the most critical of which being the capture step. Each of the steps is well described and detailed in the following Examples.

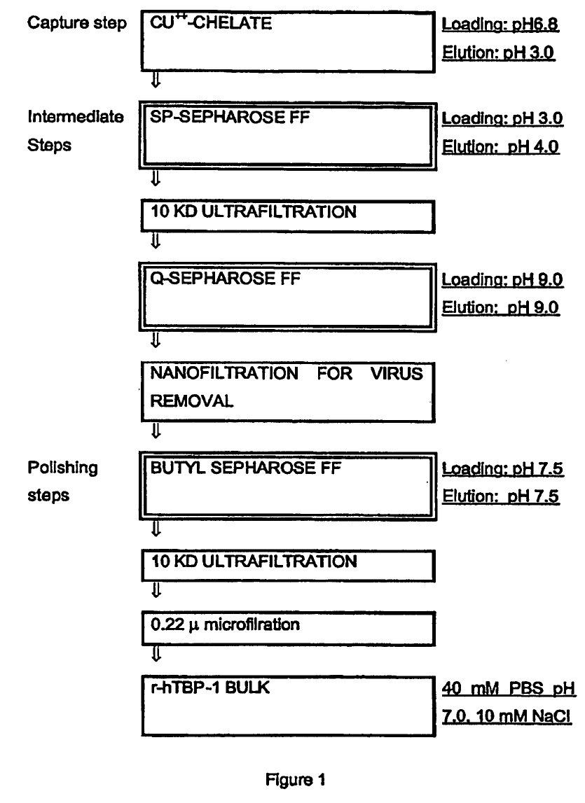

### EXAMPLES

Materials

| | |
|---|---|
| Equipment | |
| Chromatographic column XK26/20 | Pharmacia |
| (2.6 × 20 cm) | |
| Chromatographic column XK50/20 (5 × 20 cm) | Pharmacia |
| Peristaltic pump Miniplus 2 | Gilson |
| Peristaltic pump P-1 | Pharmacia |
| Chart recorder 2210 | Pharmacia |
| UV detector Uvicord 2158 | Pharmacia |
| On line pH-conductivity monitor | Biosepra |
| Low Pressure chromatographic system FPLC | Pharmacia |
| HPLC analytical system | Merck |
| Fluorimetric detector mod. 9070 | Varian |
| Refrigerated box MCF 1500 | Angelantoni |
| U.V Spectrophotometer UV1204 | Shimadzu |
| Ultrafiltration system mod. Minitan | Millipore |
| Minitan plates 4/K | Millipore |
| Stirred cell mod. 8400 | Amicon |
| Stirred cell mod. 8050 | Amicon |
| Ultrafiltration membrane type YM10 | Amicon |
| Ultrafiltration membrane type YM10 | Amicon |
| Resins and columns | |
| SP Sepharose FF | Pharmacia |
| Q Sepharose FF | Pharmacia |
| Butyl Sepharose FF | Pharmacia |
| Chelating Sepharose FF | Pharmacia |
| SP Sepharose Big Beads | Pharmacia |
| Phenyl Sepharose 6 FF (high sub) | Pharmacia |
| CM Sepharose FF | Pharmacia |
| DEAE Sepharose FF | Pharmacia |
| DEAE-HyperD | Biosepra |
| Supelcosil LC-308 0.46 × 5 | Supelco |
| Aquapore RP-300 Brownlee | Applied Biosystem |
| TSK-G2000 SW_{XL} 0.78 × 30 | TOSO-HAAS |
| Mono-Q HR 5/5 | Pharmacia |
| Chemicals | |
| Tris(hydroxymethyl)-amino methane (Tris) | Merck |
| Sodium chloride | Merck |
| Ortho-phosphoric acid 85% | Merck |
| Sodium hydroxide (pellets) | Merck |
| Di-sodium hydrogen phosphate | Merck |
| Sodium dihydrogen phosphate | Merck |
| Ethanol absolute | Merck |
| Acetonitrile (ACN) | Merck |
| Trifluoroacetic acid (TFA) | Baker |
| 50% sodium hydroxide | Baker |
| Sodium Sulphate | Merck |
| Copper sulphate | Merck |
| Zinc chloride | Merck |
| Hydrochloric acid 37% | Merck |
| 1-propanol cod. 1024 | Merck |
| Ethylenediaminotetracetic acid (EDTA) | Merck |
| Ammonium sulphate | Merck |
| Biologicals | |
| r-hTBP-1 crude harvest | INTERPHARM |
| | LABORATORIES LTD. |
| McAb to TBP-1 clone 18 | INTERPHARM |
| | LABORATORIES LTD. |
| Albumin standard cod. 2321 | Pierce |

The purification of r-hTBP-1 (Onercept) will now be described in detail.
Step 1—Capture Step
Description of Buffers and Solutions
Resin Charging Buffer

32 g of copper sulfate are dissolved in 900 ml of purified water and after dissolution the volume is brought to 1 liter.

Acidified Water

0.5 ml of acetic add is added to 1 liter of water.

Equilibration Buffer

1.68+/−0.1 ml of 85% ortho-phosphoric add and 11.68+/−0.1 g NaCl are dissolved in 900 ml of purified water, the pH is adjusted to 6.8+/−0.1 with 50% NaOH solution and the volume is brought to 1 liter.

Wash Solution

1 liter of purified water is used as washing solution.

Elution Buffer (a Range of pH 2.8 to 3.2 has Been Tested)

6.75+/−0.5 ml of 85% ortho-phosphoric acid and 5.84+/−0.1 g NaCl are dissolved in 900 ml of purified water, the pH is adjusted to 3+/−0.1 with 50% NaOH solution and the volume is brought to 1 liter. The resulting conductivity is 15+/−1 mS.

Regeneration Buffer

18.61+/−0.1 g EDTA and 58.4+/−1 g NaCl are dissolved in 900 ml of purified water and the volume is brought to 1 liter.

Sanitization Solution

40 g NaOH are dissolved in 900 ml of purified water and the volume is brought to liter.

Storage Solution

20% ethanol or 0.01M NaOH are used as storage solution.

Column Preparation

8+/−1 ml of Chelating Sepharose Fast Flow (Amersham Biosciences) is coupled with iminodiacetic acid resin and packed into the chromatographic column so that the bed height is 4+/−0.5 cm. The packed column is washed with 10 BV of acidified water and then loaded with 2 BV of 0.2 M copper sulphate pH 4-4.5. Following the manufacturer's instructions a solution 23 mM of sodium acetate pH 4-4.5 is used to facilitate the dissolution of copper sulphate and to avoid precipitation at neutral pH. The resin is then washed with 10 BV of acidified water.

Procedure

Crude harvest containing r-hTBP-1 (recombinant TNF-binding protein-1), stored at 4° C., is brought to room temperature; pH is adjusted to 6.8 by dropwise addition of 85% ortho-phosphoric acid and conductivity is brought to 21+/−1 mS by addition of solid NaCl (crude harvest can also be applied after a preliminary concentration phase of ultrafiltration to remove medium components that could negatively affect the interaction of r-hTBP-1 with copper).

The column prepared as described above is first equilibrated by flushing with 15-20 BV of equilibration buffer and then loaded with the crude harvest of r-hTBP-1 by operating at room temperature (22+/−3° C.) and at a linear flow rate of 200 ml/sqcm-hour.

The column is first washed with equilibration buffer until the UV signal reaches the baseline and then is washed with 12-15 BV of water and the column effluent is discarded.

Elution is carded out with the elution buffer and collection of eluate is started when a UV signal is detected. The elution of r-hTBP-1 is accomplished with 5-6 BV of elution buffer. The effluent containing semi-purified r-hTBP-1 is collected and stored at −20° C.

The column is regenerated with 3 BV of regeneration buffer and the column effluent is discarded. Thereafter, the column is sanitized wit 5 BV of sanitization solution.

For storage, the column is washed with 5 BV of storage solution and stored in it.

The purity data after this step are summarized in TABLE 1 below.

Performance of the Capture Step (Comparison with Zn²⁺ IMAC)

The capture step was originally carried out on a Zn²⁺-chelate IMAC column. However, the loading capacity of the capture step for crude r-hTBP-1 was considered too low (250-300 mcg r-hTBP-1 or 40 column volumes of crude harvest/ml of resin). By replacing zinc with copper, as charging metal, a significant increase in the loading capacity has been obtained. During this Cu²⁺ IMAC capture step, the r-hTBP-1 is bound to the resin, most of the contaminant proteins are eluted in the unbound fraction and semipurified r-hTBP-1 is obtained in the elution with a purity level suitable for the following steps.

By the selected conditions, the required improvement in the binding capacity has been achieved together with some other advantages. The most relevant results relative to the present invention are summarised below.

The capture step of r-hTBP-1, performed by the metal chelate chromatography, shows the following characteristics:

1. Concentration: 25-30 fold concentration of r-hTBP-1, in comparison with the crude harvest (see Table 1).

2. Purification: The step is effective in the reduction of the contaminants, as shown in Table 1.

3. Scaleability: The method is suitable for scale up and manufacturing scale;

4. Productivity: The recovery of the step is satisfactory as shown in Table 2.

Furthermore the step is very fast, reproducible and easy to be carried out. The resin can be reused after the appropriate sanitization and recharging.

Furthermore, the main advantages of the use of Cu²⁺ over Zn²⁺ can be summarised as follows:

Higher loading capacity: 1 ml of Cu-resin binds 1-42 mg of r-hTBP-1 against 0.25-0.5 mg/ml of Zn-resin;

Improvement of the purity level of material after capture step from 30-45% obtained by the Zn-resin to 40-50% of Cu-resin as shown in Table 2 (quantitative RP-HPLC).

Reduction of the number of washes step from 3 of Zn-resin to 1 Cu-resin with a reduction of working time and buffer consumption.

TABLE 1
Capture on Cu-chelate r-hTBP-1 - Recovery data by IEMA

| | | | | | % |
|---|---|---|---|---|---|
| RUN | Sample | Volume | mcg/ml | Total mg | recovery [symbol] |
| RUN 1 | Start | 1200 | 8.5 | 10.2 | — |
| | Unbound | 1300 | 1.0 | 1.3 | 12.7 |
| | Wash | 98 | 1.4 | 0.12 | 1.2 |
| | Elution | 45 | 234 | 10.5 | 100 |
| RUN 2 | Start | 1100 | 8.2 | 9.0 | — |
| | Unbound | 1200 | 0.9 | 1.0 | 11 |
| | Wash | 88 | 1.2 | 0.1 | 1.1 |
| | Elution | 38 | 214 | 8.2 | 91 |
| RUN 3 | Start | 1200 | 8.2 | 9.8 | — |
| | Unbound | 1300 | 1.6 | 2.0 | 20 |
| | Wash | 88 | 1.6 | 0.14 | 1.4 |
| | Elution | 41 | 200 | 8.2 | 83.6 |

[symbol]

calculated on the total amount of r-hTBP-1 loaded

Step 2—Ion Exchange Chromatography on SP Sepharose FF
Description of Buffers and Solutions
Equilibration Buffer

1.68 ml of 85% ortho-phosphoric acid and 17.53 g of NaCl area added to 900 ml of water with stirring, pH is adjusted to 3.0+/−0.1 with 50% NaOH and the volume is adjusted to 1 liter.

Wash Buffer

0.68 ml of 85% ortho-phosphoric acid is added to 900 ml of water, with stirring. pH is adjusted to 4.0+/−0.1 with 50% NaOH and the volume is adjusted to 1 liter.

Elution Buffer

3.37 ml of 85% orto-phosphoric acid and 17.53 g of NaCl are added to 900 ml of water, with stirring. pH is adjusted to 4.0±0.1 with 50% NaOH and the volume is adjusted to 1 liter.

Regeneration Buffer

3.37 ml of 85% orto-phosphoric add and 116.8 g of NaCl are added to 900 ml of water, with stirring. pH is adjusted to 6.00.1 with 50% NaOH and the volume is adjusted to 1 liter.

Sanitization Solution

20 g of NaOH are dissolved in 900 ml of water, with stirring and the volume is adjusted to 1 liter.

Storage Solution

200 ml of absolute ethanol are added to 800 ml of water under stirring.

Column Preparation

The column is packed with SP-Sepharose FF resin, following the manufacturer's instructions, up to 6-6.5 cm bed height.

The column is sanitized by flushing 3 BV of NaOH 0.5M followed by 3 BV of water.

The column is equilibrated by flushing 4-5 BV of equilibration buffer. pH and conductivity of column effluent are checked (pH 3.0±0.1, conductivity 29.5±0.5 mS/cm) and the column is eventually further equilibrated if the measured values are not within the indicated ranges.

NB: Alternatively, the equilibration buffer can be replaced by 25 mM Phosphate buffer pH 2.8+/−0.1 without NaCl; the wash buffer can be eliminated; the regeneration buffer can be replaced by NaCl 1.5M; and the storage solution can be replaced by 10 mM NaOH.

Procedure

All operations are performed at a temperature of 2-8° C. and at a flow rate of 40-50 ml/cm/hour.

Frozen r-hTBP-1 obtained from capture step elution is thawed either at room temperature or 6±2° C. The pH is adjusted from 3.7±0.2 to 3±0.1 by adding 85% phosphoric acid and conductivity is adjusted from 14±3 mS/cm to 22±3 mS/cm by adding solid sodium chloride and the solution is loaded on the column. After loading is completed, the column is flushed with 3 BV of equilibration buffer, followed by 4 BV of wash buffer. Alternatively, the washing with the wash buffer can be eliminated (see the NB above).

Then elution with elution buffer is started. r-hTBP-1 starts to elute after 180-220 ml. This first part is discarded and the following 3.5 BV which represent semipurified r-hTBP-1 are collected. The eluted fraction is sampled (5×0.5 ml) for IPC and stored at 6±2° C. for not more than 3 days.

After elution is completed, the column is flushed with about 3 BV of regeneration buffer. The fraction (1×1 ml) is sampled and discarded ft.

For storage, the column is flushed with 3 BV of EtOH 20% (or, alternatively with 10 mM NaOH) and stored at 6+/−2° C.

Results of seven experiments of this step are in the following TABLE 2:

TABLE 2
Performance of the cation exchange chromatograhy step -

| | Start SP | r-hTBP-1 |
|---|---|---|
| RUN | total mg | recovery |
| CS R-HTBP-1/015 RUN5 | 436 | 95.8% |
| CS R-HTBP-1/015 RUN6 | 435 | 95.4% |
| CS R-HTBP-1/015 RUN7 | 454 | 93.4% |
| CS R-HTBP-1/015 RUN8 | 419 | 93.0% |
| CS R-HTBP-1/015 RUN9 | 576 | 97.6% |
| CS R-HTBP-1/015 RUN10 | 579 | 98.7% |
| CS R-HTBP-1/015 RUN11 | 382 | 102% |

The following Table 3 shows the performance of the combination of the steps IMAC and SP-sepharose FF.

TABLE 3
Purity of r-hTBP-1 obtained from different sources

| Upstream | Purity of | | |
|---|---|---|---|
| Process | post IMAC | Purity of post SP | Source of the data |
| Serum | 58%-62% | 82%-100% | GMP Runs BS01-BS05 |
| Serum Free | 57%-77% | 81%-98% | GMP Runs MS01-MS05 |

Step 3—SP Eluate Ultrafiltration
Procedure

All operations are performed at room temperature (23±3° C.).

The ultrafilter stored in NaOH is washed with water until pH 7.0±0.5. The ultrafilter assembled with membrane is loaded with the r-hTBP-1 solution. The solution is concentrated up to 50 ml. The retentate fraction is diluted with about 200 ml of water and concentrated again to 50 ml. The washing step described above is repeated three more times.

The conductivity of the permeate is checked: if it is <0.5 mS/cm start with the following step.

If the conductivity value is >0.5 mS/cm repeat once more the present washing step.

200 ml of 50 mM Tris (at pH 9.0±0.1 and conductivity 0.55±0.1 mS/cm) are added to the retentate fraction and concentrated again up to 50 ml of solution.

The operation described above is repeated three times, and, if needed, continued until the pH and conductivity of the permeate fraction is 9.0±0.2 and 0.55±0.1 mS/cm respectively.

The retentate fraction is collected and the ultrafilter is washed with three 100 ml aliquots of 50 mM Tris (at pH 9.0±0.1 and conductivity 0.6±0.1 mS/cm) adding the washing fractions.

The ultrafilter is washed and sanitized with 0.1 M NaOH (or, alternatively, 0.5 M NaOH) by recycling for not more than 30 minutes. The ultrafilter is rinsed with water until permeate pH is 7.0±0.5. The ultrafilter is then stored in 0.01M or, alternatively, 0.05 M NaOH at 23±3° C.

Step 4—Ion Exchange Chromatography on Q-Sepharose FF

Buffers and Solutions

Equilibration buffer, 50 mM Tris pH 9.0±0.1, conductivity 0.55±0.1 mS/cm

Elution buffer 250 mM Tris pH 9.0±0.1, 50 mM NaCl conductivity 7.2±0.5 mS/cm

Regeneration buffer: 250 mM Tris pH 6.0±0.1, 2 M NaCl or, alternatively, 1.5M NaCl

Sanitization solution: 0.5M NaOH.

Storage solution: 20% Ethanol or 10 mM NaOH.

Procedure

All operations are performed in the following conditions:

Temperature: 2-8° C. or, alternatively, room temperature; Linear flow rate: 80-90 ml/cm²/hour

The pH of r-hTBP-1 post Ultrafiltration is checked and, if it is different from pH 9.0±0.1, it is adjusted with 1M Tris or 3M HCl. The conductivity is also checked.

The column is packed with Q-Sepharose FF resin, following the manufacturer's instructions, up to 13 cm bed height.

The Q-Sepharose column is then sanitized by flushing 3 BV of NaOH 0.5 M followed by 6 BV of water. Then the column is flushed with 4 BV of elution buffer and equilibrated with 78 BV of equilibration buffer, pH and conductivity of column effluent is checked (pH 9.0±0.2, conductivity 0.55±0.1 mS/cm). The equilibration of the column is eventually continuously performed if the measured values are not within the indicated ranges.

The column is then loaded with ultrafiltered r-hTBP-1 prepared as above. After loading is completed, the column is flushed with 3 BV of equilibration buffer.

Elution is started with the elution buffer. Pure h-hTBP-1 starts to elute after 1 BV; collection of r-hTBP-1 is started after the first BV according to the chromatographic profile; then elution is completed after 5-6 BV.

The column is flushed with 3 BV of regeneration buffer, sample (1×1 ml) and then discarded. The column is again flushed with 3 BV of 0.5 M NaOH, rinsed with water until the pH of the effluent is between 7 and 8. Finally the column is flushed with 3 BV of EtOH 20% and stored at 24-C.

Step 5—Nanofiltration on DV 50 PALL

The stainless steel support is installed in the disc-holder and the DV50 filter (47 mm diameter) is placed on the support Pall Uldpor® VF Grade DV50 is a filter cartridge which is normally used for viruses removal. A few drops of water are added on the top of the disk. The appropriate seals are installed and the disc-holder is closed tightly. The system is filled with 50 ml of Q elution buffer, closed and connected to the Nitrogen source.

At the beginning of the flushing the nitrogen is opened at an initial pressure of 0.5 bar and then the vent valve located on the disc-holder is opened in order to purge the system.

As soon as the first drop of liquid appears at the vent valve on the disc-holder, it is closed tightly and the nitrogen is opened to the right pressure, 3.0-3.5 bar.

The membrane is then flushed with all the 50 ml of buffer, in order to assure that the membrane is wet and to eliminate air, if present, between the sheets of the membrane and perform the integrity test on the filter.

The system is filled with material coming from the previous step and operated as follows: at the beginning of the filtration the nitrogen is opened at an initial pressure of 0.5 bar and then the vent valve located on the disc-holder is opened in order to purge the system. As soon as the first drop of solution starts to appears, the vent valve of the disc-holder is closed and the nitrogen opened to a pressure of 1.5-2.5 bar.

The nitrogen pressure is kept at 1.5-2.5 bar and then the solution is filtered.

The filtered solution is collected in a container and at the end of the filtration, the nitrogen source is dosed and the vent valve is opened to eliminate excess of nitrogen.

At the end of the filtration, the system is washed with 5-10 ml of the elution buffer of the previous step, at the same working pressure of 1.5-2.5 bar.

The washing solution is collected in the same container of the filtered solution and sampled for IPC.

Step 6—Hydrophobic Interaction on Butyl Sepharose FF

Buffers and Solutions

Equilibration buffer: 200 mM Tris-HCl pH 7.5±0.1, 1 M Na₂SO4 conductivity 90±5 mS/cm

Elution buffer 200 mM Tris-HCl pH 7.5±0.1, 0.7 M Na₂SO4, conductivity 75±5 mS/cm

Regeneration solution: Purified water

Sanitization solution: 1M NaOH

Storage solution: 20% ethanol or 10 mM NaOH

Procedure

All operations are performed at a temperature of 23±3° C. and at a linear flow rate of 80-90 ml/cm/hour. Solid Na₂SO₄ is added to Q-Sepharose eluate, post 100 KD Ultrafiltration under stirring, up to 1M. After that the dissolution of the salt is completed, the pH is adjusted to 7.5±0.1 with 3M HCl. The column is then flushed with 3 BV of NaOH 1M followed by 4 BV of purified water.

The column is again flushed with 5-6 BV of equilibration buffer. The pH and conductivity of effluent (pH 7.5±0.2, conductivity 90±5 mS/cm) are checked and the column equilibration is continuously performed, if measured values are out of indicated ranges.

The solution prepared as above is loaded on to the column and, after loading is completed, the column is washed with 3 BV of equilibration buffer. Wash with equilibration buffer is continued.

After 2-3 BV of wash, proteins start to elute. This fraction contains r-hTBP-1, 10-12% about of total, contaminated by cell culture contaminants. This wash is prolonged until protein elution reaches the plateau giving a broad peak (about 2 BV).

Then elution is started with elution buffer. The first 1-2 BV are pooled with the washing sample, since it contains a small amount of contaminants and immediately thereafter collection of r-hTBP-1 is started.

Purified r-hTBP-1 elutes immediately after the contaminated material and elution is continued for another 2.5-3 BV. The collection is stopped when the UV absorbance reaches the 0.5% of max. After collection of r-hTBP-1, the fraction (5×0.5 ml) is sampled and stored it at 2-8° C. for not more than 3 days.

The column is flushed with 3 BV of purified water and the fraction collected.

The column is sanitized with 3 BV of 1 M NaOH and rinsed with water until the pH of effluent is between 7 and 8.

Then the column is flushed again with three column volume of ethanol 20% and stored at room temperature for not more than 2 weeks.

Step 7—10 KD Ultrafiltration

The stirred cell type 8400, assembled with the membrane, is loaded with the Butyl-Sepharose eluate. The solution is concentrated to about 25 ml, under nitrogen pressure of 3 bars. The retentate fraction is diluted with about 100 ml of water and concentrated again to 25 ml. The washing step described above is repeated three further times. The conductivity of the permeate is checked: If it is <0.3 mS/cm then the following step can be started. If the conductivity value is >0.3 mS/cm, the washing step should be repeated.

100 ml of bulk buffer is added to the retentate fraction and concentrated again up to 25 ml of solution. This operation is repeated three times, and, if needed, until the pH and conductivity of the permeate fraction is 7.1±02 and 5.8±0.2 MS/cm, respectively.

The retentate fraction is discarded and loaded on the smaller ultrafiltration stirred cell type 8050, assembled with the membrane. The retentate is concentrated to minimum volume (about 3-5 ml). The retentate fraction is collected and the ultrafilter with bulk is washed by adding the washing fractions to the concentrated r-hTBP-1. The final volume is adjusted in order to obtain a final concentration of about 20-30 mg/ml by OD 280 nm (ε=0.71).

The ultrafilters are washed and sanitized with 0.2 M NaOH by recycling for at least 30 minutes. The ultrafilters are then rinsed with water until the permeate pH is 7.0±0.5. The ultrafilters are then stored in NaOH 0.01M at 6±2° C.

Step 8—Microfiltration

A disposable syringe is connected to a 0.22 it filter, filled with the r-hTBP-1 concentrated solution, filtered and washed twice with 1 ml of bulk buffer by pooling the washes with the filtered bulk. The resulting solution is sampled for analytical tests (15×0.2 ml) and stored at −20° C.

Results are satisfactory under the quantification and purity points of view as shown by the following tables (Tables 4 to 6) reflecting the results of an adequate number of replications of this process (RUN).

Most critical to the process of this invention is the initial chromatography step on Cu⁺² chelate column. Moreover, it is also important the combination of the SP Sepharose chromatography at an add pH with a following Q Sepharose at a basic pH. In these conditions, strikingly good results have been obtained by subjecting a crude harvest from CHO production of r-hTBP-1 (Onercept). The capture step in particular has been shown to be able to 25-30 fold concentrate r-hTBP-1, to effectively reduce contaminants, to have a satisfactory recovery of the protein and to be scaleable for industrial manufacturing.

Even more surprising is the fact that outstanding purity data are obtained both when the starting material is a crude supernatant from serum-containing cell culture and when it comes from serum-free cultures, as will be shown below.

TABLE 4
Step and cumulative recovery data

| | SP-Sepharose | Q-Sepharose | Butyl | Bulk | |
|---|---|---|---|---|---|
| | Step | Step | Step | Step | |
| | Recovery | Recovery | Recovery | Recovery | Overall |
| Run | (%) | (%) | (%) | (%) | Yield * |
| RUN 1 | 95.8 | 98.2 | 84.8 | 102 | 73.8 |
| RUN 2 | 95.4 | 90.4 | 86.2 | 104 | 79.5 |
| RUN 3 | 93.4 | 94.3 | 90.4 | 106 | 82.3 |
| RUN 4 | 93.0 | 93.3 | 90.5 | 102 | 89 |
| RUN 5 | 97.6 | 95.7 | 80.9 | 108 | 83.3 |
| RUN 6 | 98.7 | 89.2 | 87.3 | 101 | 80.1 |
| RUN 7 | 102 | 90 | 81.6 | 100 | 75.2 |

TABLE 5
Bulk quantitation data

| | | | Quantitative | | |
|---|---|---|---|---|---|
| Bulk | Volume | O.D. | RP-HPLC | Bradford | Biol. activity |
| batch | (ml) | (mg/ml) | (mg/ml) | (mg/ml) | (IU/mg) [symbol] |
| RUN 1 | 16 | 20.3 | 20.2 | 22.7 | 25985 |
| RUN 2 | 13.7 | 25 | 25.3 | 26.2 | 27350 |
| RUN 3 | 14 | 26 | 26.7 | 26.1 | 23834 |
| RUN 4 | 13.5 | 28.6 | 27.7 | 30.2 | 23003 |
| RUN 5 | 16 | 29 | 30.2 | 28.7 | 23803 |
| RUN 6 | 16 | 29 | 29 | 27.0 | 27339 |
| RUN 7 | 13 | 20.5 | 20.4 | 19.6 | 27752 |

[symbol]

mg of r-hTBP-1 obtained by OD

TABLE 6
Bulk Purity data

| | | Cell | | | SDS-PAGE |
|---|---|---|---|---|---|
| | Purity by | Culure | Fluorimetric | | Silver |
| Bulk | SE-HPLC | Proteins | RP-HPLC | DNA | Stained |
| batch | (%) | (ppm) [symbol] | (ppm) [symbol] | (pg/mg) [symbol] | (ppm) ¤- [symbol] |
| RUN 1 | 99.7 | <6 | <95 | 17 | <100 ppm |
| RUN 2 | 99.9 | <5 | <75 | 10 | <100 ppm |
| RUN 3 | 99.9 | 3 | <46 | 11 | <100 ppm |
| RUN 4 | 99.7 | <4 | <65 | 12 | <100 ppm |
| RUN 5 | 99.7 | <4 | <86 | 11.5 | <100 ppm |
| RUN 6 | 99.7 | <2 | <39 | n.d. | <100 ppm |
| RUN 7 | 99.9 | n.d. | <121 | n.d. | <100 ppm |

By applying analogous process steps to the other TNF receptor, r-hTBP-2, similar quantitation and purity data are obtained.
Analytical Protocols
1. Quantitative RP-HPLC—Working Procedure

The following method has been used to quantitative the r-hTBP-1 in all purification samples. It employes a CB column with acqueous TFA and n-propanol; a good resolution between r-hTBP-1 and cell culture contaminants is obtained. The r-hTBP-1 can be resolved in one or two peaks depending on the column batch. The procedure is described here below.

1.1 Equipment and Materials and Method

Analytical HPLC System (Merck or equivalent)
Dynamic mixer (Merck or equivalent)
Column SUPELCOSIL LC-308 Ø 0.46×5 cm-cod 5-8851-Supelco
Eluent A: 0.1% aqueous TFA
Eluent B: 0.1% TFA in water/n-propanol 50:50
Eluent C: Acetonitrile
Temperature: 23±3° C.
UV Detection: 214 nm
Injection time: 62 minutes
Injection volume: 10-100 μl
Standard: BTC10, 1.53 mg/ml by OD 280 nm (ε=0.71) Injected at 10 and 20 μl

Gradient:

| | Flow rate | Time | | | |
|---|---|---|---|---|---|
| Step | ml/min | (minutes) | % A | % B | % C |
| 1 | 0.7 | 0 | 90 | 10 | 0 |
| 2 | 0.7 | 5 | 70 | 30 | 0 |
| 3 | 0.7 | 14 | 65 | 35 | 0 |
| 4 | 0.7 | 27 | 0 | 100 | 0 |
| 5 | 0.7 | 35 | 0 | 100 | 0 |
| 6 | 1 | 35.1 | 0 | 20 | 80 |
| 7 | 1 | 40 | 0 | 20 | 80 |
| 8 | 1 | 40.1 | 90 | 10 | 0 |
| 9 | 1 | 50 | 90 | 10 | 0 |
| 10 | 0.7 | 61 | 90 | 10 | 0 |

1.2 Calculation

The amount of r-hTBP-1 in each purification sample has been obtained as follows:

calculate the response factor (RF) for the standard (BTC10) according to the formula:

$$RF = \frac{TBP 1 mcg \text{/} ml}{TBP 1 peak area}$$

Multiply the r-hTBP-1 peak area of each sample by the RF of the standard obtaining the concentration of the sample in mcg/ml as shown:
TBP1 mcg/ml=TBP1 peak area×RF standard

Please note that

The BTC 10 used as standard has been chosen on the basis of availability;

The retention time of r-hTBP-1 peak can shift at each new buffer preparation (1-3 min);

Concentrated sample has to be diluted in eluant A.

2. Fluorimetric RP-HPLC—Working Procedure

Based on previous experiences with other recombinant proteins a RP-HPLC analysis with a fluorimetric detection has been set up to estimate the purity level of the residual cell culture contaminants both in r-hTBP-1 bulks and in in process samples since no immunochemical method was available when the purification study started.

This method was found useful to monitor the removal of cell culture contaminants in the last purification step, i.e. Butyl Sepharose chromatography and it was determinant in the selection of the operative conditions of the above step, since it could be used to analyze the in-process samples and no special materials and/or apparatus are required. The RP-HPLC is fast (run time 62 minutes) and gives results comparable to the immunoassay when this test became available. Since a standard for contaminants was not yet available, a BSA solution from Pierce was used as standard to estimate the contamination level in the samples. As the quantitative RP-HPLC, this test gives a good resolution between r-hTBP-1 and BSA area.

2.1 Equipment, Materials and Method

Analytical HPLC System (Merck or equivalent)
Dynamic mixer
Fluorimetric detector (Varian or equivalent)
Column: Aquapore RP-300, 7μ, Brownlee, Ø0.46×22 cm-cod 0711-0059, Applied Biosystem
Eluent A: 0.1% aqueous TFA
Eluent B: 0.1% TFA in Acetonitrile
Temperature: 23±3° C.
λ extraction: 220 nm
λ emission: 330 nm
Injection volume: 10-100 μl
Injection time: 62 minutes
Standard: BSA (Pierce) 2 mg/ml diluted 1:100, 10 and 20 μl injected;
Control: BTC10, 1.53 mg/ml by OD 280 nm (ε=0.71), as it is 200 μl injected;
r-hTBP-1 samples: 1-5 mg/ml by OD 280 nm (ε=0.71).

Gradient

| | Flow rate | Time | | |
|---|---|---|---|---|
| Step | ml/min | (minutes) | % A | % B |
| 1 | 2 | 0 | 70 | 30 |
| 2 | 2 | 5 | 70 | 30 |
| 3 | 2 | 15 | 65 | 35 |
| 4 | 2 | 25 | 50 | 50 |
| 5 | 2 | 35 | 50 | 50 |
| 6 | 2 | 36 | 0 | 100 |
| 7 | 2 | 45 | 0 | 100 |
| 8 | 2 | 46 | 70 | 30 |
| 9 | 2 | 61 | 70 | 30 |

2.2 Calculation

The amount of contaminants in each Butyl purification sample is obtained as follows:

calculate the response factor (RF) for the standard (BSA) according to the formula:

$$RF = \frac{BSA mcg injected}{BSA peak area}$$

Multiply the contaminants peaks area of each sample by the RF of the standard and by 1000 obtaining the amount of contaminants in the sample injected in ng. Dividing this value by the amount of r-hTBP-1 injected the contamination in parts per million is obtained, according to the formula:

$$ppm contaminants = \frac{contaminants peak areas \times RF BSA \times 1000}{TBP 1 mg injected}$$

Please note that:

Test sample has to be diluted in eluant A.

The contamination of the control sample ranges between 190 and 240 ppm.

3. Analysis and Characterization of the r-hTPB-1 Bulk

The analytical methods described hereinafter have been set up and used to characterize the r-hTBP-1 bulk originated by the new purification procedure.

3.1 SE-HPLC

This method was developed with the aim to quantitate the amount of dimers and aggregates in the final bulk. The method can discriminate between r-hTBP-1 monomer and its dimer and/or aggregates. This has been proved by testing some r-hTBP-1 samples after UV treatment, a method widely known to generate aggregate forms of molecules. Briefly the method is carried out as follows:

3.1.1 Equipment, Materials and Method,

| | |
|---|---|
| Equipment: | Analytical HPLC System |
| Column: | TSK G2000 SW_{XL} cod. 08021 (TosoHaas) |
| Mobile phase: | 0.1M Sodium phosphate pH 6.7, 0.1M sodium sulfate |
| Temperature: | 23 ± 3° C. |
| UV detection: | 214 nm |
| Injection volume: | 10-100 μl corresponding to 20-30 mcg of r-hTBP-1 |
| | (by OD) |
| Injection time: | 30 minutes |
| Standard: | BTC10, 1.53 mg/ml by OD 280 nm (ε = 0.71) |
| | 10-20 μl injected |
| r-hTBP-1 bulk: | diluted to 1-2 mg/ml by OD 280 nm (ε = 0.71) |
| | 10-20 μl injected |

The purity of the sample is expressed as % of purity of r-hTBP-1 peak/total area ratio.
3.2 IE-HPLC

This method was developed to evaluate the isoform composition in the final bulk with the aim to replace the chromatofocussing technique generally used for the above purpose. In contrast to the chromatofocussing, the IEC analysis is more advantageous because is faster than the above, requires less material (150-200 mcg instead of 1-2-mg), employes common buffers and does not require pretreatment of the test sample. Since r-hTBP-1 is a glycoprotein, as a substance of that nature, it is characterized by different isoforms having each one a different isoelectric point that determines a different behaviour when tested by an ion exchange analysis. 12 different peaks, each one corresponding to a glycosilation variant, are obtained. By the present method all the isoforms of the r-hTBP-1 have been isolated and fully characterized. Briefly the method is carried out as follows:

3.2.1 Equipment, Materials and Method

Analytical inert HPLC System

| | |
|---|---|
| Column: | Mono Q HR 5/5 |
| Buffer A: | 40 mM Tris/HCl pH 8.5 |
| Buffer B: | 40 mM Tris/HCl pH 8.5, 0.3 M NaCl |

Gradient:

| | Flow rate | Time | | |
|---|---|---|---|---|
| Step | ml/min | (minutes) | % A | % B |
| 1 | 1 | 0 | 100 | 0 |
| 2 | 1 | 10 | 90 | 10 |
| 3 | 1 | 30 | 75 | 25 |
| 4 | 1 | 40 | 65 | 35 |
| 5 | 1 | 41 | 0 | 100 |
| 6 | 1 | 51 | 0 | 100 |
| 7 | 1 | 52 | 100 | 0 |
| 8 | 1 | 70 | 100 | 0 |

| | |
|---|---|
| Flow rate: | 1 ml/min |
| Temperature: | 23 ± 3° C. |
| UV detection: | 220 nm |
| Injection amount: | 10-15 mcl corresponding to 150-200 mcg of |
| | r-hTBP-1(by OD) |
| Injection time: | 70 minutes |
| Sample: | r-hTBP-1 bulk and reference diluted 1:2 with purified |
| | water |

4. Quantitation of r-hTBP-1 by OD

The concentration of the r-hTBP-1 bulks produced in accordance with the present invention was determined by optical density at 280 nm using the molar extinction coefficient (e) calculated in house on r-hTBP-1 bulk produced during the initial phase of the purification of r-hTBP-1. Three representative r-hTBP1 bulks produced with the new purification process have been used, obtaining ε=0.776. This new extinction coefficient will be used for the scale up and production phases. Since the concentration of the bulks is in the range of 20-30 mg/ml, it is necessary dilute the material to 1 mg/ml with bulk buffer (40 mM PBS pH 7.1±0.2, 10 mM NaCl), prior to test the absorbance at 280 nm.

5. Protein Determination by Bradford

The Bradford method was used to quantitate total proteins in the r-hTBP-1 bulk (see Bradford, M M. Analytical Biochemistry 72: 248-254, 1976 and Stoscheck, C M. Methods in Enzymology 182: 50-69, 1990). The standard used in this test is BSA.

6. In Vitro Bioassay

The bioactivity of r-hTBP-1 consists in its capacity to bind TNF c. This test was used to assay both the in process samples and bulks.

1. Process for the isolation of pure TNF-binding proteins comprising eluting a crude solution of a TNF-binding protein on an Immobilized Metal Affinity Chromatography (IMAC) using copper as metal.

2. Process for the purification of recombinant TNF-binding proteins, comprising, as capture step, an Immobilized Metal Affinity Chromatography using copper as metal.

3. The process according to claims 1 or 2, wherein the elution from the IMAC column is carried out at a pH comprised between 2.8 and 3.2.

4. The process according to any preceding claims, wherein the elution from the IMAC column is carried out at salinity comprised between 14 to 16 mS.

5. The process according to any preceding claim, which further comprises the following steps, as intermediate steps: an Ion Exchange Chromatography (IEC) at an acidic pH, preferably between 3 and 4, followed by an ion exchange chromatography at a basic pH, preferably between 8 and 10.

6. The process according to any preceding claim, which further comprises, as polishing step, a Hydrophobic Interaction Chromatography (HIC).

7. The process according to any preceding claim, wherein each chromatography step is followed by an ultrafiltration step.

8. The process according to any preceding claim, wherein the TNF-binding protein is recombinant h-TBP-1.

9. Process for the manufacture of a TNF-binding protein comprising isolating or purifying the protein according to the process of anyone of the preceding claims.
